(19) United States
(12) Patent Application Publication
(10) Pub. No.: US 2002/0192251 A1
(43) Pub. Date: Dec. 19, 2002

(54) Cosmetic composition containing a mixture of fibers

(75) Inventor: Nathalie Collin, Sceaux (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/109,731

(22) Filed: Apr. 1, 2002

(30) Foreign Application Priority Data
Mar. 30, 2001 (FR) 01 04392

(51) Int. Cl. A61K 9/70

(52) U.S. Cl. 424/401; 424/443

(57) ABSTRACT

A composition such as a mascara containing a physiologically acceptable aqueous medium, cellulose fibrils and a second fiber other than cellulose fibrils. The composition makes it possible to obtain good maintenance of the fibers on keratinous substances. Making up and caring for keratinous substances.

### BACKGROUND OF THE INVENTION

1. Field of the Invention

The present invention relates to a composition comprising, preferably in a physiologically aqueous medium, a mixture of fibers. The invention composition is intended in particular for the cosmetics field. The invention also relates to a process for making up or cosmetically caring for keratinous substances. The composition and the make-up or care process according to the invention are more particularly intended for human keratinous substances, such as the skin (including the lips), nails or keratinous fibers, in particular substantially longitudinal keratinous fibers, such as the eyelashes, eyebrows and hair. More especially, the invention relates to a mascara.

The composition according to the invention can be provided in any form including in the form of a composition for coating the eyelashes (in particular a mascara), an eyeliner, a product for the eyebrows, a product for the lips, a face powder, an eyeshadow, a foundation, a product for making up the body, a concealer, a nail varnish, a product for caring for the skin, including the scalp, or a product for the hair (hair mascara).

2. Discussion of the Background

The use of fibers in make-up products is known, in particular for their lengthening effects in mascaras (see JP-A-57/158714, JP-A-3/153613), their moisturizing properties in lipsticks (see U.S. Pat. No. 5,498,407), for improving the outline of a lipstick on the edges of the lips (see EP-A-0 106 762), for repairing broken nails (see FR-A-1 529 329), or else in products for caring for the skin, for their velvety feel (see JP-A-7/196440).

However, after the application of these compositions to keratinous substances, the fibers do not adhere well to the keratinous substances: they become detached and are removed from the keratinous substances over time. The removal of these fibers then results in a perceptible decline in the desired cosmetic properties introduced by the fibers (for example, loss of the lengthening effect of the mascara), making it necessary to repeat the application of the product. Furthermore, for a mascara, the fibers, on becoming detached from the eyelashes, fall onto the eyelids and the cheeks, giving an unattractive appearance; the detached fibers can also become lodged in the eyes and can result in eye discomfort or ocular intolerance problems.

One aim of the present invention is to make available a cosmetic composition which makes it possible to overcome the above-mentioned disadvantages, comprising fibers which adhere well to keratinous substances.

### SUMMARY OF THE INVENTION

The inventor has now found, surprisingly, that the use of cellulose fibrils in a composition comprising fibers other than cellulose fibrils, makes it possible to obtain good maintenance of the fibers deposited on keratinous substances. This combination of fibers adhere well to keratinous substances and do not become detached therefrom over time. Thus, the cosmetic properties of the composition and in particular of the make-up are satisfactorily retained over time. In particular, when the composition is a mascara, it is found that the lengthening effect of the mascara lasts over time, due to the good maintenance of the fibers on the eyelashes. The fibers hold well to the eyelashes and do not fall off onto the eyelids or cheeks or into the eyes, which avoids the problems of unattractiveness or eye discomfort.

### DETAILED DESCRIPTION OF THE INVENTION

More specifically, one subject of the invention is a composition comprising, preferably in a physiologically acceptable aqueous medium, a first fiber chosen from cellulose fibrils and a second fiber different from the first fiber.

Another subject of the invention is a cosmetic process for making up or caring for human keratinous substances comprising the application, to the keratinous substances, of a composition as defined above.

A further subject of the invention is the use of a composition as defined above for producing good hold of the fibers to keratinous substances.

Another subject of the invention is the use of cellulose fibrils in a composition, in particular a mascara, comprising, in a cosmetically acceptable aqueous medium, fibers other than the cellulose fibrils, for producing good hold of the fibers on keratinous substances, in particular the eyelashes.

Another subject of the invention is the use of a mascara comprising a composition as defined above for lengthening the eyelashes and/or obtaining good hold of the fibers to the eyelashes.

The composition according to the invention most preferably comprises a physiologically acceptable medium comprising an aqueous phase. The term “physiologically acceptable medium” is understood to mean a medium which is nontoxic and which is capable of being applied to the skin, superficial body growths or lips of human beings, such as a cosmetic medium.

The composition according to the invention comprises a first fiber chosen from cellulose fibrils.

In the present application, the term “cellulose fibrils” is understood as meaning both nanofibrils and microfibrils, the length of which is less than 100 μm. A preferred length is less than 50 μm. These fibrils generally have a length of greater than 1 μm and preferably ranging from 5 to 40 μm. In addition, the cellulose fibrils can have a diameter ranging, for example, from 1 to 100 nm (0.001 to 0.1 μm); the length/diameter ratio can be equal to or greater than 30.

The cellulose fibrils used according to the invention are preferably amorphous, that is to say that they preferably exhibit a degree of crystallinity of less than or equal to 50%, preferably ranging from 15 to 50%.

In addition, the cellulose fibrils used according to the invention are not limited and can be obtained by mechanical or chemical extraction from plants or algae or by bacterial fermentation, for example. Furthermore, they can be provided in the form of dry matter or as a dispersion, in particular an aqueous dispersion.

The cellulose fibrils used according to the invention can be provided as such or modified. Thus, they can be mixed with an additive and in particular with carboxylated cellulose, as disclosed in the documents WO-A-98/02486 and WO-A-98/02487. They can also be in the modified form and, for example, they can be modified by carboxylic acids, as disclosed, for example, in the document EP-A-726 356, and/or be used in combination with a polyhydroxylated organic compound, as disclosed, for example, in the document FR-A-2 769 836.

Use may in particular be made, as cellulose fibrils, of those sold under the names “cellulon” by Kelco, those sold under the name “Fibriliance” by Soliance and those from Rhodia, in particular that referred to hereinafter as “compound A”, comprising 85% of nanofibrils and 15% of carboxymethylcellulose, and obtained according to Example 1 of Application FR-2 769 836.

The cellulose fibrils are generally introduced into the aqueous phase of the composition.

The composition of the invention can comprise an amount of cellulose fibrils ranging from 0.01 to 5% by weight, preferably from 0.1 to 3% and better still from 0.5 to 2% by weight, with respect to the total weight of the composition, including all amounts and subranges therebetween, such as 0.7, 0.9, 1, 1.3, 1.5, 1.8, and 2.5%.

The composition also comprises a second fiber, referred to as the additional or second fiber, that is different from the cellulose fibrils (first fiber) described above.

The term “fiber” should be understood as meaning an object with a length (or greater length) L and a diameter D such that L is greater than D, D being the diameter of the circle in which the cross section of the fiber is framed. In particular, the L/D ratio (or form factor) is preferably chosen within the range from 3.5 to 2,500, preferably from 5 to 500 and better still from 5 to 150.

The second fibers which can be used in the composition of the invention can be fibers of synthetic or naturaland inorganic or organic origin. They can be short or long, individual or organized, for example plaited or braided, and hollow or solid. They can have any shape and can in particular be circular or polygonal (square, hexagonal or octagonal) in cross section, according to the specific application envisaged. In particular, their ends may be blunted and/or polished to prevent injury.

The second fibers have a length (or greater length) of greater than or equal to 100 μm. A preferred length is from 100 μm to 10 mm, more preferably ranging from 0.1 mm to 5 mm and better still ranging from 1 mm to 3.5 mm. Their cross section can preferably be included within a circle with a diameter ranging from 2 nm to 500 μm, preferably ranging from 100 nm to 100 μm and better still from 1 μm to 50 μm. The weight or count of the fibers is often given in denier or decitex and represents the weight in grams per 9 km of yarn. Preferably, the second fibers according to the invention have a count chosen within the range from 0.15 to 30 denier and better still from 0.18 to 18 denier.

The second fibers include those used in the manufacture of textiles and in particular silk fiber, cotton fiber, wool fiber, flax fiber, cellulose fibers (particularly extracted in particular from wood, vegetables or algae), rayon fiber, polyamide (Nylon®) fiber, viscose fiber, acetate fiber, in particular rayon acetate fiber, poly(p-phenylene terephthalamide) (or aramide) fiber, in particular Kevlar® fiber, acrylic polymer fiber, in particular poly(methyl methacrylate) or poly(2-hydroxyethyl methacrylate) fiber, polyolefin fiber and in particular polyethylene or polypropylene fiber, glass fiber, silica fiber, carbon fiber, in particular in the graphite form, polytetrafluoroethylene (such as Teflon®) fiber, insoluble collagen fiber, polyester fiber, poly(vinyl chloride) fiber, poly(vinylidene chloride) fiber, poly(vinyl alcohol) fiber, polyacrylonitrile fiber, chitosan fiber, polyurethane fiber, poly(ethylene phthalate) fiber, or fibers formed from a blend of polymers, such as those mentioned above, for example polyamide/polyester fibers.

Use may also be made of fibers used in surgery, such as resorbable synthetic fibers prepared from glycolic acid and ε-caprolactone (Monocryl from Johnson & Johnson); resorbable synthetic fibers of the type which is a copolymer of lactic acid and of glycolic acid (Vicryl from Johnson & Johnson); poly(terephthalic ester) fibers (Ethibond from Johnson & Johnson) and stainless steel threads (Acier from Johnson & Johnson), in particular for a nail varnish application.

Furthermore, the second fibers may or may not be surface treated and may or may not be coated. Coated fibers which can be used in the invention include polyamide fibers coated with copper sulphide for antistatic effect (for example, R-STAT from Rhodia) or another polymer which makes possible a specific arrangement of the fibers (specific surface treatment) or a surface treatment which induces color/hologram effects (Lurex fiber from Sildorex, for example).

Use is preferably made of fibers of synthetic origin and in particular of organic fibers, such as those used in surgery. Use may advantageously be made of water-insoluble fibers.

The second fibers which can be used in the composition according to the invention are preferably polyamide, cellulose, poly(phenylene terephthalamide) or polyethylene fibers. Their length (L) can range from 0.1 to 5 mm, preferably from 0.25 to 3 mm, and their mean diameter can range from 1 to 50 μm. Use may in particular be made of the polyamide fibers sold by Etablissements P. Bonte under the name Polyamide 0.9 Dtex 3 mm, having a mean diameter of 6 μm, a count of approximately 0.9 Dtex and a length ranging from 0.3 mm to 5 mm. Use may also be made of poly(p-phenylene terephthalamide) fibers with a mean diameter of 12 μm and a length of approximately 1.5 mm, such as those sold under the name of Kevlar Floc by Du Pont Fibers, or else of cellulose (or rayon) fibers having a mean diameter of 50 μm and a length ranging from 0.5 mm to 6 mm, such as those sold under the name of Natural Rayon Flock Fiber RC1BE-N003-M04 by Claremont Flock. Use may also be made of polyethylene fibers, such as those sold under the name of Shurt Stuff 13 099 F by Mini Fibers.

The second fibers can be present in the composition according to the invention in a content ranging from 0.01 to 10% by weight with respect to the total weight of the composition, preferably from 0. 1% to 5% by weight and better still from 0.3% to 2% by weight, including all values and subranges therebetween such as 0.5, 1 and 1.5%.

The first and second fibers can advantageously be present in the composition according to the invention according to a second fiber/first fiber (cellulose fibrils) ratio by weight ranging from 0.8 to 2.5 and better still ranging from 1 to 2, including all values and subranges therebetween such as 1.1, 1.3, 1.5, 1.7 and 1.9.

The aqueous medium of the composition can comprise, consist, or consist essentially of water and optionally of a water-miscible solvent (miscibility in water greater than 50% by weight at 25° C.), such as lower monoalcohols having from 1 to 5 carbon atoms, such as ethanol or isopropanol, glycols having from 2 to 8 carbon atoms, such as propylene glycol, ethylene glycol, 1,3-butylene glycol or dipropylene glycol, C₃-C₄ ketones or C₂-C₄ aldehydes.

The water, and optionally the water-miscible organic solvent, can be present in a content ranging from 1% to 95% by weight with respect to the total weight of the composition, preferably from 5% to 80% by weight and better still from 10% to 60% by weight, including all values and subranges therebetween.

The composition according to the invention can additionally comprise one or more waxes. The term “wax” is understood to mean, within the meaning of the present invention, a lipophilic fatty compound which is solid at ambient temperature (25° C.) and atmospheric pressure (760 mm of Hg, i.e. 10⁵ Pa), with a reversible solid/liquid state change, having a melting temperature of greater than 30° C. and better still of greater than 55° C. which can range up to 200° C., in particular up to 120° C. The melting point values correspond, according to the invention, to the melting peak measured using a differential scanning calorimeter (D.S.C.), for example the calorimeter sold under the name DSC 30 by Metler, with a temperature rise of 5 or 10° C. per minute.

By bringing a wax to its melting temperature, it is possible to render it miscible with an oil and to form a microscopically homogeneous mixture but, by bringing the temperature of the mixture back to ambient temperature, recrystallization of the wax in the oil of the mixture is obtained.

The waxes useful in the invention include those generally used in the cosmetics and dermatological fields. Mention may in particular be made of beeswax, lanolin wax and Chinese insect waxes; rice wax, carnauba wax, candelilla wax, ouricury wax, sugar cane wax, Japan wax and sumac wax; montan wax, microcrystalline waxes, paraffin waxes, ozokerites, ceresin wax, lignite wax, polyethylene waxes, waxes obtained by the Fischer-Tropsch synthesis, or fatty acid esters and glycerides which are solid at 40° C. and better still at more than 55° C.

Waxes obtained by catalytic hydrogenation of animal or vegetable oils having linear or branched C₈-C₃₂ fatty chains are also included. Mention may in particular be made, among these, of hydrogenated jojoba oil, hydrogenated sunflower oil, hydrogenated castor oil, hydrogenated coconut oil and hydrogenated lanolin oil.

Also included are silicone waxes and fluorinated waxes.

The waxes present in the invention composition can be dispersed in the form of particles in an aqueous medium. These particles can have a mean size ranging from 50 nm to 10 μm and preferably from 50 nm to 3.5 μm. The wax can in particular be present in the form of a waxes-in-water emulsion, it being possible for the waxes to be in the form of particles with a mean size ranging from 1 μm to 10 μm and preferably from 1 μm to 3.5 μm.

In another embodiment of the composition according to the invention, the wax can be present in the form of a wax microdispersion, the wax being in the form of particles with a mean size of less than 1 μm and ranging in particular from 50 nm to 500 nm. Wax microdispersions are disclosed in documents EP-A-5 571 196 and EP-A-1 048 282.

The wax can also exhibit a hardness ranging from 0.05 MPa to 15 MPa and preferably ranging from 6 MPa to 15 MPa. The hardness is determined by the measurement of the compressive force, measured at 20° C. using the texture analyser sold under the name TA-XT2i by Rheo, equipped with a stainless steel cylinder with a diameter of 2 mm moving at the measurement speed of 0.1 mm/s and penetrating into the wax to a depth of penetration of 0.3 mm. To carry out the hardness measurement, the wax is melted at a temperature equal to the melting point of the wax +20° C. The molten wax is poured into a receptacle with a diameter of 30 mm and a depth of 20 mm. The wax is recrystallized at ambient temperature (25° C.) for 24 hours and then the wax is stored for at least 1 hour at 20° C. before carrying out the hardness measurement. The value of the hardness is the compressive force measured divided by the surface area of the cylinder of the texture analyser in contact with the wax.

The wax can be present in the composition according to the invention in a content ranging from 0.1% to 50% by weight with respect to the total weight of the composition, preferably from 0.5% to 30% by weight and better still from 1% to 20% by weight, including all values and subranges therebetween, such as 3, 5, 8, 10, 12, 15, 17 and 19%.

The composition according to the invention can comprise one or more volatile organic oils or solvents and/or one or more nonvolatile oils.

The term “volatilie organic oil or solvent” is understood to mean, within the meaning of the invention, any nonaqueous medium capable of evaporating on contact with the skin in less than one hour, at ambient temperature and atmospheric pressure. The volatile organic solvent or solvents and the volatile oils of the invention are volatile cosmetic oils and organic solvents which are liquid at ambient temperature and which have a non-zero vapour pressure, at ambient temperature and atmospheric pressure, ranging in particular from 10⁻² to 300 mm of Hg (1.33 Pa to 40 000 Pa) and preferably of greater than 0.3 mm of Hg (30 Pa). The term “nonvolatile oil” is understood to mean an oil which remains on the skin at ambient temperature and atmospheric pressure for at least several hours and which has in particular a vapour pressure of less than 10⁻² mm of Hg (1.33 Pa).

These oils can be hydrocarbonaceous oils, silicone oils, fluorinated oils or their mixtures.

The term “hydrocarbonaceous oil” is understood to mean an oil mainly comprising hydrogen and carbon atoms and optionally oxygen, nitrogen, sulphur or phosphorus atoms. The volatile hydrocarbonaceous oils can be chosen from hydrocarbonaceous oils having from 8 to 16 carbon atoms and in particular branched C₈-C₁₆ alkanes, such as C₈-C₁₆ isoalkanes of petroleum origin (also known as isoparaffins), for example isododecane (also known as 2,2,4,4,6-pentamethylheptane), isodecane or isohexadecane, and for example the oils sold under the trade names of Isopars or Permetyls, branched C₈-C₁₆ esters, isohexyl neopentanoate, and their mixtures. Other volatile hydrocarbonaceous oils, such as oil distillates, in particular those sold under the name Shell Solt by Shell, can also be used. Preferably, the volatile solvent is chosen from volatile hydrocarbonaceous oils having from 8 to 16 carbon atoms and their mixtures.

Use may also be made, as volatile oils, of volatile silicones, such as, for example, volatile linear or cyclic silicone oils, in particular those having a viscosity ≦8 centistokes (8×10⁻⁶ m²/s), having in particular from 2 to 7 silicon atoms, these silicones optionally comprising alkyl or alkoxy groups having from 1 to 10 carbon atoms. Mention may in particular be made, as volatile silicone oil which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Use may also be made of volatile fluorinated solvents, such as nonafluoromethoxybutane or perfluoromethylcyclopentane.

The volatile oil can be present in the composition according to the invention in a content ranging from 0.1% to 98% by weight with respect to the total weight of the composition, preferably from 1% to 65% by weight, including all values and subranges therebetween.

The composition can also comprise at least one nonvolatile oil chosen in particular from nonvolatile hydrocarbonaceous and/or silicone and/or fluorinated oils.

Useful nonvolatile hydrocarbonaceous oil include:

hydrocarbonaceous oils of vegetable origin, such as triglycerides composed of esters of fatty acids and of glycerol, the fatty acids of which can have various chain lengths from C₄ to C₂₄, it being possible for these chains to be linear or branched and saturated or unsaturated; these oils are in particular wheat germ, sunflower, grape seed, sesame, maize, apricot, castor, karite, avocado, olive, soybean, sweet almond, palm, rapeseed, cottonseed, hazelnut, macadamia, jojoba, alfalfa, poppy, pumpkinseed, sesame, cucumber, rapeseed, blackcurrant seed, evening primrose, millet, barley, quinoa, rye, safflower, candlenut, passionflower or musk rose oil; or triglycerides of caprylic/capric acids, such as those sold by the company Stearineries Dubois or those sold under the names Miglyol 810, 812 and 818 by Dynamit Nobel,

synthetic ethers having from 10 to 40 carbon atoms;

linear or branched hydrocarbons of mineral or synthetic origin, such as liquid petrolatum, polydecenes, hydrogenated polyisobutene, such as parleam, squalane, and their mixtures;

synthetic esters, such as oils of formula R₁COOR₂ in which R₁ represents the residue of a linear or branched fatty acid comprising from 1 to 40 carbon atoms and R₂ represents a hydrocarbonaceous chain, in particular a branched hydrocarbonaceous chain, comprising from 1 to 40 carbon atoms, provided that R₁+R₂ is ≧10, such as, for example, Purcellin oil (cetearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, or octanoates, decanoates or ricinoates of alcohols or of polyalcohols, such as propylene glycol dioctanoate; hydroxylated esters, such as isostearyl lactate or diisostearyl malate; and pentaerythritol esters;

fatty alcohols which are liquid at ambient temperature with a branched and/or unsaturated carbonaceous chain having from 12 to 26 carbon atoms, such as octyldodecanol, isostearyl alcohol, oleyl alcohol, 2-hexyldecanol, 2-butyloctanol or 2-undecylpentadecanol;

higher fatty acids, such as oleic acid, linoleic acid or linolenic acid; and their mixtures.

Nonvolatile silicone oils which can be used in the composition according to the invention include nonvolatile polydimethylsiloxanes (PDMSs), polydimethylsiloxanes comprising pendant alkyl or alkoxy groups and/or alkyl or alkoxy groups at the end of the silicone chain, which groups each have from 2 to 24 carbon atoms, or phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Fluorinated oils which can be used in the invention include fluorosilicone oils, fluorinated polyethers or fluorinated silicones as disclosed in the document EP-A-847 752.

The nonvolatile oils can be present in the composition according to the invention in a content ranging from 0.1% to 80% by weight, preferably from 0.1% to 50% by weight, with respect to the total weight of the composition, and better still from 0.1% to 20% by weight, including all values and subranges therebetween.

The aqueous medium of the composition can comprise, consist of, or consists essentially of water and optionally of a water-miscible solvent (miscibility in water greater than 50% by weight at 25° C.), such as lower monoalcohols having from 1 to 5 carbon atoms, such as ethanol or isopropanol, glycols having from 2 to 8 carbon atoms, such as propylene glycol, ethylene glycol, 1,3-butylene glycol or dipropylene glycol, C₃-C₄ ketones or C₂-C₄ aldehydes.

The water, and optionally the water-miscible organic solvent, can be present in a content ranging from 1% to 95% by weight with respect to the total weight of the composition, preferably from 5% to 80% by weight and better still from 10% to 60% by weight, including all values and subranges therebetween.

The composition according to the invention can comprise emulsifying surface-active agents present preferably in a proportion ranging from 2 to 30% by weight with respect to the total weight of the composition and better still from 5% to 15%. These surface-active agents can be chosen from anionic or nonionic surface-active agents. Reference may be made to the document “Encyclopedia of Chemical Technology, Kirk-Othmer”, volume 22, p. 333-432, 3rd edition, 1979, Wiley, for the definition of the properties and functions (emulsifying) of surfactants, in particular p. 347-377 of this reference for anionic and nonionic surfactants.

The surfactants preferably used in the composition according to the invention are chosen:

from nonionic surfactants: fatty acids, fatty alcohols, polyethoxylated or polyglycerolated fatty alcohols, such as polyethoxylated stearyl or cetearyl alcohols, esters of a fatty acid and of sucrose, alkyl glucose esters, in particular polyoxyethylenated fatty esters of C₁-C₆ alkyl glucose, and their mixtures;

from anionic surfactants: C₁₆-C₃₀ fatty acids neutralized with amines, aqueous ammonia or alkaline salts, and their mixtures.

Use is preferably made of surfactants which make it possible to obtain an oil-in-water or wax-in-water emulsion.

The composition according to the invention can comprise at least one film-forming polymer.

The film-forming polymer can be a polymer dissolved or dispersed in the form of solid particles in an aqueous phase of the composition or else a polymer dissolved or dispersed in the form of solid particles in a liquid fatty phase. The composition can comprise a blend of these polymers. When the film-forming polymer is provided in the form of solid particles, these particles can exhibit a mean particle size ranging from 5 nm to 600 nm and preferably from 20 nm to 300 nm.

The film-forming polymer can be present in the composition according to the invention in a content on a dry basis ranging from 0.1% to 60% by weight with respect to the total weight of the composition, preferably from 0.5% to 40% by weight and better still from 1% to 30% by weight.

In the present application, the term “film-forming polymer” is understood to mean a polymer capable of forming, by itself alone or in the presence of a subsidiary agent which is able to form a film, a continuous film which adheres to a support, in particular to keratinous substances.

Use is preferably made of a film-forming polymer capable of forming a hydrophobic film, that is to say a polymer, the film of which has a solubility in water at 25° C. of less than 1% by weight.

Mention may be made, among film-forming polymers which can be used in the composition of the present invention, of synthetic polymers, of radical type or of polycondensate type, polymers of natural origin, and their blends.

The term “radical film-forming polymer” is understood to mean a polymer obtained by polymerization of monomers comprising unsaturation, in particular ethylenic unsaturation, each monomer being capable of homopolymerizing (unlike polycondensates).

The film-forming polymers of radical type can in particular be vinyl polymers or copolymers, in particular acrylic polymers.

The vinyl film-forming polymers can result from polymerization of monomers comprising ethylenic unsaturation having at least one acid group and/or esters of these acid monomers and/or amides of these acid monomers.

Use may be made, as monomer carrying an acid group, of α,β-ethylenic unsaturated carboxylic acids, such as acrylic acid, methacrylic acid, crotonic acid, maleic acid or itaconic acid. Use is preferably made of (meth)acrylic acid and crotonic acid and more preferably of (meth)acrylic acid.

The esters of acid monomers are advantageously chosen from esters of (meth)acrylic acid (also known as (meth)acrylates), in particular alkyl (meth)acrylates, especially C₁-C₃₀ alkyl (meth)acrylates, preferably C₁-C₂₀ alkyl (meth)acrylates, aryl (meth)acrylates, in particular C₆-C₁₀ aryl (meth)acrylates, or hydroxyalkyl (meth)acrylates, in particular C₂-C₆ hydroxyalkyl (meth)acrylates.

Special mention may be made, among alkyl (meth)acrylates, of methyl methacrylate, ethyl methacrylate, butyl methacrylate, isobutyl methacrylate, 2-ethylhexyl methacrylate, lauryl methacrylate or cyclohexyl methacrylate.

Special mention may be made, among hydroxyalkyl (meth)acrylates, of hydroxyethyl acrylate, 2-hydroxypropyl acrylate, hydroxyethyl methacrylate or 2-hydroxypropyl methacrylate.

Special mention may be made, among aryl (meth)acrylates, of benzyl acrylate and phenyl acrylate.

The esters of (meth)acrylic acid which are particularly preferred are the alkyl (meth)acrylates.

According to the present invention, the alkyl group of the esters can be either fluorinated or perfluorinated, that is to say that a portion or all of the hydrogen atoms of the alkyl group are substituted by fluorine atoms.

Special mention may for example be made, as amides of the acid monomers, of (meth)acrylamides and in particular N-alkyl(meth)acrylamides, especially with a C₂-C₁₂ alkyl. Mention may be made, among N-alkyl(meth)acrylamides, of N-ethylacrylamide, N-(t-butyl)acrylamide, N-(t-octyl)acrylamide and N-undecylacrylamide.

The vinyl film-forming polymers can also result from the homopolymerization or copolymerization of monomers chosen from vinyl esters and styrene monomers. In particular, these monomers can be polymerized with acid monomers and/or their esters and/or their amides, such as those mentioned above.

Special mention may be made, as examples of vinyl esters, of vinyl acetate, vinyl neodecanoate, vinyl pivalate, vinyl benzoate and vinyl t-butylbenzoate.

Special mention may be made, as styrene monomers, of styrene and α-methylstyrene.

It is possible to use any monomer known to a person skilled in the art coming within the categories of acrylic and vinyl monomers (including monomers modified by a silicone chain).

Special mention may be made, among film-forming polycondensates, of polyurethanes, polyesters, polyesteramides, polyamides, and epoxyester resins, or polyureas.

The polyurethanes can be chosen from polyurethanes which are anionic, cationic, nonionic or amphoteric, polyurethane-acrylics, polyurethane-polyvinylpyrrolidones, polyester-polyurethanes, polyether-polyurethanes, polyureas, polyurea-polyurethanes, and their blends.

The polyesters can be obtained, in a known way, by polycondensation of dicarboxylic acids with polyols, in particular diols.

The dicarboxylic acid can be aliphatic, alicyclic or aromatic. Mention may be made, as examples of such acids, of: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers can be used alone or as a combination of at least two dicarboxylic acid monomers. Preferably, among these monomers, phthalic acid, isophthalic acid or terephthalic acid is chosen.

The diol can be chosen from aliphatic, alicyclic or aromatic diols. Use is preferably made of a diol chosen from: ethylene glycol, diethylene glycol, triethylene glycol, 1,3-propanediol, cyclohexanedimethanol or 4-butanediol.

Use may be made, as other polyols, of glycerol, pentaerythritol, sorbitol or trimethylolpropane.

The polyesteramides can be obtained analogously to the polyesters by polycondensation of diacids with diamines or aminoalcohols. Use may be made, as diamine, of ethylenediamine, hexamethylenediamine or meta- or paraphenylenediamine. Use may be made, as aminoalcohol, of monoethanolamine.

The polyester can additionally comprise at least one monomer carrying at least one —SO₃M group, with M representing a hydrogen atom, an ammonium ion NH₄⁺ or a metal ion, such as, for example, an Na⁺, Li⁺, K⁺, Mg²⁺, Ca²⁺, Cu²⁺, Fe²⁺ or Fe³⁺ ion. Use may in particular be made of a bifunctional aromatic monomer comprising such an —SO₃M group.

The aromatic nucleus of the bifunctional aromatic monomer additionally carrying an —SO₃M group as described above can be chosen, for example, from benzene, naphthalene, anthracene, diphenyl, oxydiphenyl, sulphonyldiphenyl or methylenediphenyl nuclei. Mention may be made, as examples of bifunctional aromatic monomer additionally carrying an —SO₃M group, of: sulphoisophthalic acid, sulphoterephthalic acid, sulphophthalic acid or 4-sulphonaphthalene-2,7-dicarboxylic acid.

It is preferable to use copolymers based on isophthalate/sulphoisophthalate and more particularly copolymers obtained by condensation of diethylene glycol, cyclohexanedimethanol, isophthalic acid and sulphoisophthalic acid. Such polymers are sold, for example, under the Eastman AQ® trademark by Eastman Chemical Products.

The optionally modified polymers of natural origin can be chosen from shellac resin, sandarac gum, dammars, elemis, copals, cellulose polymers, and their mixtures.

According to a preferred embodiment of the composition according to the invention, the film-forming polymer can be present in the form of particles in aqueous dispersion, a form generally known under the name of latex or pseudolatex. The techniques for the preparation of these dispersions are well known to a person skilled in the art.

Aqueous dispersions of film-forming polymer include acrylic dispersions sold under the names Neocryl XK-90®, Neocryl A-1070®, Neocryl A-1090®, Neocryl BT-62®, Neocryl A-1079® or Neocryl A-523® by Avecia-Neoresins, Dow Latex 432® by Dow Chemical or Daitosol 5000 AD® by Daito Kasey Kogyo; or alternatively of the aqueous polyurethane dispersions sold under the names Neorez R-981® or Neorez R-974® by Avecia-Neoresins, Avalure UR-405®, Avalure UR-410®, Avalure UR-425®, Avalure UR-450®, Sancure 875®, Sancure 861®, Sancure 878® or Sancure 2060® by Goodrich, Impranil 85® by Bayer or Aquamere H-1511® by Hydromer.

Use may also be made, as aqueous dispersion of film-forming polymer, of the dispersions of polymers resulting from the radical polymerization of one or more radical monomers inside and/or partially at the surface of preexisting particles of at least one polymer chosen from the group consisting of polyurethanes, polyureas, polyesters, polyesteramides and/or alkyds. These polymers are generally known as hybrid polymers.

According to another embodiment of the composition according to the invention, the film-forming polymer can be a water-soluble polymer and is thus present in the aqueous phase of the composition in the dissolved form. Examples of water-soluble film-forming polymers include:

proteins, such as proteins of plant origin, such as wheat or soya proteins; or proteins of animal origin, such as keratins, for example keratin hydrolysates and sulphonic keratins;

anionic, cationic, amphoteric or nonionic chitosan or chitin polymers;

cellulose polymers, such as hydroxyethylcellulose, hydroxypropylcellulose, methylcellulose, ethylhydroxyethylcellulose or carboxymethylcellulose, and quaternized cellulose derivatives;

acrylic polymers or copolymers, such as polyacrylates or polymethacrylates;

vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; or poly(vinyl alcohol);

optionally modified polymers of natural origin, such as:

gums arabic, guar gum, xanthan derivatives or karaya gum;

alginates and carrageenans;

glycoaminoglycans, hyaluronic acid and its derivatives;

shellac resin, sandarac gum, dammars, elemis or copals;

deoxyribonucleic acid;

mucopolysaccharides, such as hyaluronic acid, chondroitin sulphates, and their mixtures.

According to another embodiment of the composition according to the invention, the film-forming polymer can be present in a liquid fatty phase comprising organic oils or solvents, such as those described above. The term “liquid fatty phase” is understood to mean, within the meaning of the invention, a fatty phase which is liquid at ambient temperature (25° C.) and atmospheric pressure (760 mm of Hg, i.e. 10⁵ Pa) and which is composed of one or more fatty substances which are liquid at ambient temperature, also known as oils, and which are generally compatible with one another.

The liquid fatty phase preferably comprises a volatile oil, optionally as a mixture with a nonvolatile oil, it being possible for the oils to be chosen from the abovementioned oils.

According to another embodiment of the composition according to the invention, the film-forming polymer can be present in the form of surface-stabilized particles dispersed in the liquid fatty phase.

The dispersion of surface-stabilized polymer particles can be manufactured as disclosed in the document EP-A-749 747.

The polymer particles may be surface-stabilized by virtue of a stabilizing agent which can be a block polymer, a graft polymer and/or a random polymer, alone or in a blend.

Dispersions of film-forming polymer in the liquid fatty phase, in the presence of stabilizing agents, are disclosed in particular in the documents EP-A-749 746, EP-A-923 928 and EP-A-930 060, the contents of which are incorporated by way of reference in the present application.

The size of the particles of polymers in dispersion either in the aqueous phase or in the liquid fatty phase can range from 5 nm to 600 nm and preferably from 20 nm to 300 nm, including all values and subranges therebetween.

According to another embodiment of the composition according to the invention, the film-forming polymer can be dissolved in the liquid fatty phase; it is then said that the film-forming polymer is a fat-soluble polymer.

Examples of fat-soluble polymers include copolymers of vinyl ester (the vinyl group being connected directly to the oxygen atom of the ester group and the vinyl ester having a saturated, linear or branched, hydrocarbonaceous radical of 1 to 19 carbon atoms bonded to the carbonyl of the ester group) and of at least one other monomer which can be a vinyl ester (other than the vinyl ester already present), an α-olefin (having from 8 to 28 carbon atoms), an alkyl vinyl ether (the alkyl group of which comprises from 2 to 18 carbon atoms) or an allyl or methallyl ester (having a saturated, linear or branched, hydrocarbonaceous radical of 1 to 19 carbon atoms bonded to the carbonyl of the ester group).

These copolymers can be crosslinked using crosslinking agents which can be either of the vinyl type or of the allyl or methallyl type, such as tetraallyloxyethane, divinylbenzene, divinyl octanedioate, divinyl dodecanedioate and divinyl octadecanedioate.

Examples of these copolymers include the copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene, crosslinked with 0.2% of divinylbenzene, and allyl propionate/allyl stearate, crosslinked with 0.2% of divinylbenzene.

Mention may also be made, as fat-soluble film-forming polymers, of fat-soluble homopolymers and in particular those resulting from the homopolymerization of vinyl esters having from 9 to 22 carbon atoms or of alkyl acrylates or methacrylates, the alkyl radicals having from 10 to 20 carbon atoms.

Such fat-soluble homopolymers can be chosen from poly(vinyl stearate), poly(vinyl stearate) crosslinked using divinylbenzene, diallyl ether or diallyl phthalate, poly(stearyl (meth)acrylate), poly(vinyl laurate) or poly(lauryl (meth)acrylate), it being possible for these poly(meth)acrylates to be crosslinked using ethylene glycol or tetraethylene glycol dimethacrylate.

The fat-soluble copolymers and homopolymers defined above are known and are disclosed in particular in Application FR-A-2 262 303; they can have a weight-average molecular weight ranging from 2,000 to 500,000 and preferably from 4,000 to 200,000.

Mention may also be made, as fat-soluble film-forming polymers which can be used in the invention, of polyalkylenes and in particular copolymers of C₂-C₂₀ alkenes, such as polybutene, alkylcelluloses with a saturated or unsaturated, linear or branched, C₁ to C₈ alkyl radical, such as ethylcellulose and propylcellulose, or vinylpyrrolidone (VP) copolymers and in particular copolymers of vinylpyrrolidone and of a C₂ to C₄₀ alkene and better still a C₃ to C₂₀ alkene. Mention may be made, by way of examples of VP copolymers which can be used in the invention, of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

The composition according to the invention can comprise a subsidiary agent which is able to form a film which promotes the formation of a film with the film-forming polymer. Such an agent which is able to form a film can be chosen from any compound known to a person skilled in the art as being capable of fulfilling the desired role and in particular can be chosen from plasticizing agents and coalescence agents.

The composition according to the invention can also comprise a coloring material, such as pulverulent coloring materials, fat-soluble dyes or water-soluble dyes. This coloring material can be present in a content ranging from 0.01% to 50% by weight with respect to the total weight of the composition, preferably ranging from 0.01% to 30% by weight.

The pulverulent coloring materials can be chosen from pigments and pearlescent agents.

The pigments can be white or colored, inorganic and/or organic and coated or uncoated. Mention may be made, among inorganic pigments, of titanium dioxide, which is optionally surface treated, zirconium, zinc or cerium oxides and iron or chromium oxides, manganese violet, ultramarine blue, chromium hydrate and ferric blue. Mention may be made, among organic pigments, of carbon black, pigments of D & C type and lakes based on cochineal carmine or on barium, strontium, calcium or aluminium.

The pearlescent agents can be chosen from white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

The fat-soluble dyes are, for example, Sudan red, D&C Red 17, D&C Green 6, β-carotene, soybean oil, Sudan brown, D&C Yellow 11, D&C Violet 2, D&C Orange 5, quinoline yellow or annato. The water-soluble dyes are, for example, beetroot juice or methylene blue.

The composition of the invention can additionally comprise any additive conventionally used in cosmetics, such as antioxidants, fillers, preservatives, fragrances, neutralizing agents, thickeners, cosmetic or dermatological active principles, such as, for example, emollients, moisturizers, vitamins or sunscreens, and their mixtures. These additives can be present in the composition in a content ranging from 0.01 to 20% of the total weight of the composition and better still from 0.01 to 10% (if present).

Of course, a person skilled in the art will take care to choose the optional additional additives and/or their amount so that the advantageous properties of the composition according to the invention are not, or not substantially, detrimentally affected by the envisaged addition.

The composition according to the invention can be manufactured by known processes used generally in the cosmetics or dermatological field, all within the skill of the ordinary artisan in view of this disclosure.

The invention is illustrated in more detail in the following non-limiting examples. AM means active material (no solvent, etc.).

### EXAMPLE 1

A mascara having the following composition was prepared:

| | | |
|---|---|---|
| Carnauba wax | 2.6 | g |
| Beeswax | 3.3 | g |
| Paraffin wax | 10.3 | g |
| Hydrogenated jojoba oil | 0.2 | g |
| Hydrogenated palm oil | 0.2 | g |
| 2-Amino-2-methyl-1,3-propanediol | 0.8 | g |
| Triethanolamine | 2.4 | g |
| Stearic acid | 6.6 | g |
| Poly(sodium methacrylate) (Darvan 7 | 0.25 | g AM |
| from Vanderbilt) | | |
| Hydroxyethylcellulose crosslinked with | 0.1 | g |
| epichlorohydrin quaternized with | | |
| trimethylamine (JR 400 from Union | | |
| Carbide) | | |
| Ethyl acrylate/methyl methacrylate | 2.5 | g AM |
| (80/20) copolymer as a 50% AM aqueous | | |
| dispersion (Daitosol 5000 AD from Saito) | | |
| Polyamide fibers (3 mm long and 0.9 Dtex, | 1 | g |
| from the company Paul Bonte) | | |
| Cellulose microfibrils (Cellulon from | 0.5 | g |
| Monsanto Kelco) | | |
| Black iron oxide | 7 | g |
| Preservatives | q.s. | |

| | | |
|---|---|---|
| Water | q.s. for 100 | g |

This mascara is easily applied and adheres well to the eyelashes, during and after application.

The make-up obtained confers a lengthening effect on the eyelashes and the hold of the polyamide fibers on the eyelashes is very good.

### EXAMPLE 2

A mascara having the following composition was prepared:

| | | |
|---|---|---|
| Cellulose microfibrils | 0.17 | g |
| (Cellulon from Monsanto Kelco) | | |
| Water | 9.3 | g |
| Polyamide fibers (3 mm long and 0.9 Dtex, | 1 | g |
| from the company Paul Bonte) | | |
| Carnauba wax | 4.6 | g |
| Hydrogenated jojoba oil | 2.2 | g |
| Paraffin | 2.2 | g |
| Beeswax | 8.0 | g |
| Rice starch | 0.8 | g |
| Bentonite | 7 | g |
| Vinyl acetate/allyl stearate (65/35) | 2.0 | g |
| copolymer (Mexomère PQ from Chimex) | | |
| Poly(vinyl laurate) (Mexomère PP | 0.7 | g |
| from Chimex) | | |
| Propylene carbonate | 2.3 | g |
| Pigments | 4.6 | g |
| Preservatives | q.s. | |

| | | |
|---|---|---|
| Isododecane | q.s. for 100 | g |

This waterproof mascara gives very good elongation to the eyelashes and the polyamide fibers hold very well to the eyelashes throughout the day.

Obviously, numerous modifications and variations of the present invention are possible in light of the above teachings. It is therefore to be understood that within the scope of the appended claims, the invention may be practiced otherwise than as specifically described herein.

French patent application 0104392 filed Mar. 30, 2001, is incorporated herein by reference, as are all documents, patents, articles, publications and references mentioned herein.

Those of ordinary skill are able to make and use the invention compositions as claimed below in view of this disclosure.

1. A composition comprising a physiologically acceptable aqueous medium, cellulose fibrils, and a second fiber that is not cellulose fibrils.

2. The composition according to claim 1, wherein the cellulose fibrils have a length of less than 50 μm.

3. The composition according to claim 1, wherein the cellulose fibrils have a length ranging from 5 to 40 μm.

4. The composition according to claim 1, wherein the cellulose fibrils have a diameter ranging from 1 to 100 nm.

5. The composition of claim 1, wherein the cellulose fibrils have a length/diameter ratio of greater than 30.

6. The composition of claim 1, wherein the cellulose fibrils are mixed with carboxylated cellulose and/or are modified by carboxylic acids and/or are present in combination with a polyhydroxylated organic compound.

7. The composition according to claim 1, wherein the cellulose fibrils are present in an amount ranging from 0.01 to 5% by weight with respect to the total weight of the composition.

8. The composition of claim 1, wherein the second fiber has a length ranging from 100 μm to 10 mm.

9. The composition according to claim 1, wherein the second fiber is selected from the group consisting of silk fiber, cotton fiber, wool fiber, flax fiber, cellulose fibers, polyamide fiber, rayon fiber, viscose fiber, acetate fiber, poly(p-phenylene terephthalamide) fiber, acrylic polymer fiber, polyolefin fiber glass fiber, silica fiber, carbon fiber, polytetrafluoroethylene fiber, insoluble collagen fiber, polyester fiber, poly(vinyl chloride) fiber, poly(vinylidene chloride) fiber, poly(vinyl alcohol) fiber, polyacrylonitrile fiber, chitosan fiber, polyurethane fiber, poly(ethylene phthalate) fiber, and fibers formed from blends of polymers.

10. The composition according to claim 1, wherein the second fiber is a fiber of synthetic origin.

11. The composition according to claim 1, wherein the second fiber is a polyamide, poly(p-phenylene terephthalamide), cellulose or polyethylene fiber.

12. The composition according to claim 1, wherein the second fiber has a length L and a diameter D such that L/D is within the range from 5 to 500.

13. The composition according to claim 1, wherein the second fiber has a count within the range from 0.15 to 30 denier.

14. The composition according to claim 1, wherein the second fiber has a cross section included within a circle with a diameter ranging from 2 nm to 500 μm.

15. The composition according to claim 1, wherein the second fiber is present in an amount ranging from 0.01% to 10% by weight with respect to the total weight of the composition.

16. The composition according to claim 1, further comprising a wax.

17. The composition according to claim 16, wherein said wax has a melting temperature of from greater than 30° C. up to 120° C.

18. The composition according to claim 16, wherein said wax is selected from the group consisting of beeswax, lanolin wax, Chinese insect waxes, rice wax, carnauba wax, candelilla wax, ouricury wax, cork fiber wax, sugar cane wax, Japan wax, sumac wax, montan wax, microcrystalline waxes, paraffin waxes, ozokerites, ceresin wax, lignite wax, polyethylene waxes, waxes obtained by the Fischer-Tropsch synthesis, fatty acid esters and glycerides which are solid at 40° C., waxes obtained by catalytic hydrogenation of animal or vegetable oils having linear or branched C₈-C₃₂ fatty chains, silicone waxes and fluorinated waxes.

19. The composition according to claim 16, wherein said wax has a hardness ranging from 0.05 MPa to 15 MPa.

20. The composition according to claim 16, wherein the wax is present in an amount ranging from 0.1% to 50% by weight with respect to the total weight of the composition.

21. The composition according to claim 1, further comprising a volatile organic oil or solvent.

22. The composition according to claim 21, comprising a volatile hydrocarbonaceous oil having from 8 to 16 carbon atoms.

23. The composition according to claim 21, wherein the volatile oil is present in an amount ranging from 0.1% to 98% by weight with respect to the total weight of the composition.

24. The composition according to claim 1, further comprising a nonvolatile oil.

25. The composition according to claim 24, comprising a nonvolatile oil selected from the group consisting of hydrocarbonaceous oils of mineral, vegetable or synthetic origin, synthetic esters or ethers, silicone oils and their mixtures.

26. The composition according to claim 1, wherein the aqueous medium comprises water and a water-miscible organic solvent.

27. The composition according to claim 1, comprising water in an amount ranging from 1 to 95% by weight with respect to the total weight of the composition.

28. The composition according to claim 1, further comprising a film-forming polymer.

29. The composition according to claim 28, wherein the film-forming polymer is selected from the group consisting of vinyl polymers, polyurethanes, polyesters, polyamides, polyureas and cellulose polymers.

30. The composition according to claim 28, wherein the film-forming polymer is dissolved in an aqueous phase or is in the form of particles in aqueous dispersion.

31. The composition according to claim 28, wherein the film-forming polymer is dissolved or dispersed in the form of surface-stabilized particles in a liquid fatty phase.

32. The composition according to claim 28, wherein the film-forming polymer is present in an amount ranging from 0.1% to 60% by weight with respect to the total weight of the composition.

33. The composition according to claim 1, further comprising at least one coloring material.

34. The composition according to claim 33, wherein the coloring material is selected from the group consisting of pigments, pearlescent agents, fat-soluble dyes and water-soluble dyes.

35. The composition according to claim 33, wherein the coloring material is present in an amount of 0.01 to 50% of the total weight of the composition.

36. The composition according to claim 1, wherein said composition constitutes a composition for caring for or for making up keratinous substances.

37. The composition according to claim 36, further comprising at least one additive selected from the group consisting of antioxidants, fillers, preservatives, fragrances, neutralizing agents, thickeners, and cosmetic or dermatological active principles.

38. The composition according to claim 36, in the form of a mascara, an eyeliner, a product for the eyebrows, a product for the lips, a face powder, an eyeshadow, a foundation, a product for making up the body, a concealer, a nail varnish, a product for caring for the skin or a product for the hair.

39. Mascara comprising a composition according to claim 1.

40. The composition of claim 1, wherein the cellulose fibrils have a length ranging from 5 to 40 μm,
wherein the cellulose fibrils have a diameter ranging from 1 to 100 nm,
wherein the cellulose fibrils have a length/diameter ratio of greater than 30,
wherein the cellulose fibrils are present in an amount ranging from 0.01 to 5% by weight with respect to the total weight of the composition,
wherein the second fiber has a length ranging from 100 μm to 10 mm,
wherein the second fiber has a length L and a diameter D such that L/D is within the range from 5 to 500,
wherein the second fiber has a cross section included within a circle with a diameter ranging from 2 nm to 500 μm, and
wherein the second fiber is present in an amount ranging from 0.01% to 10% by weight with respect to the total weight of the composition.

41. A process for making up or caring for human keratinous substances, comprising the application, to the keratinous substances, of a composition according to claim 36.
